(19) United States
(12) Patent Application Publication
(10) Pub. No.: US 2002/0028466 A1
(43) Pub. Date: Mar. 7, 2002

(54) Fast scale-up using solid phase chemistry

(76) Inventors: Stephen P. Raillard, Mountain View, CA (US); Gongjun Ji, Santa Clara, CA (US); Adam D. Mann, Menlo Park, CA (US); Ted A. Baer, Palo Alto, CA (US)

Correspondence Address: Affymax Research Institute, 4001 Miranda Avenue, Palo Alto, CA, 94304, US

(21) Appl. No.: 09/849,547

(22) Filed: May 4, 2001

Related U.S. Application Data
(63) Continuation of application No. 09/187,857, filed on Nov. 6, 1998.

(51) Int. Cl. C07C 69/76; G01N 33/53; G01N 33/543

(52) U.S. Cl. 435/7.1; 436/518; 560/111

(57) ABSTRACT

A novel approach using solid phase chemistry for scale-up was developed. The method makes use of high-load Merrifield resin.

### BACKGROUND OF THE INVENTION

The present invention is related to the area of chemical synthesis. More specifically, one embodiment of the present invention provides methods for large scale production of chemical compounds using solid phase chemistry.

The emergence of combinatorial chemistry has profoundly changed the way in which drug discovery is conducted in the pharmaceutical industry. See, Brown (1996) Mol. Div. 217. Particularly the combination of solid phase synthesis with high-throughput screening promises to generate lead molecules in a dramatically shortened time. As a consequence chemical development departments will be asked to scale-up more compounds, faster than ever, in the quest to find novel drug substances.

The attractiveness of solid-phase chemistry can be attributed to the elimination of time-consuming work-up steps and the fact that reactions can be driven to completion by using large excesses of reagents. These features make solid-phase chemistry potentially attractive also for chemical development purposes. In addition, it would be beneficial if the development work could capitalize on the initial research effort in designing and optimizing a particular solid-phase strategy. Unfortunately, due to their high costs the popular resins generally employed in solid phase and combinatorial chemistry are too expensive to be of interest for scale-up purposes.

The present invention meets these needs by providing methods for the large scale preparation of chemical compounds using solid phase chemistry.

### SUMMARY OF THE INVENTION

The present invention provides new methods for the large scale (i.e., multigram) preparation of chemical compounds using solid phase chemistry. More specifically, these methods utilize Merrifield resin as the solid phase support.

Another embodiment provides for the use of 2,6-dichlorobenzoylchloride as a coupling agent in ester synthesis.

A further embodiment provides for the use of dioxane as a cosolvent with water in the preparation of hydroxylmethylresin.

A further understanding of the nature and advantages of the inventions herein may be realized by reference to the remaining portions of the specification.

### DESCRIPTION OF THE PREFERRED EMBODIMENT

I. Terminology

Unless otherwise stated, the following terms used in the specification and claims have the meanings given below:

“Linker” refers to a molecule or group of molecules attached to a solid support and spacing a synthesized compound from the solid support, such as for exposure/binding to a receptor.

“Merrifield resin” refers to a chloromethylpolystyrene-divinylbenzene resin which can be produced either by co-polymerization of sytrene, divinylbenzene and p-cloromethylstyrene or by chloromethylation of polystyrene. See Bodanszky et al. in “Peptide Synthesis”, E. Grass and J. Meienhofer (Eds.), Academic Press, Y. Wiley, New York, 1976 and G. Barany et al. (1980) Peptides 2:1.

“Sasrin resin” refers to generally to a 2-methoxy-4-alkoxybenzyl alcohol resin. See U.S. Pat. Nos. 4,831,084 and 4,914,151. Sasrin resin can be prepared from Merrifield resin as described hereinbelow.

“Solid support”, “support”, and “substrate” refer to a material or group of materials having a rigid or semi-rigid surface or surfaces. The solid support(s) will take the form of beads, resins, gels, microspheres, or other geometric configurations. A particularly preferred support is Merrifield resin.

“Wang resin” refers generally to a p-benzyloxybenzyl alcohol resin and more specifically cross-linked polystyrene beads functionalized with TFA labile p-benzyloxybenzyl alcohol functionalities. See Wang (1973) J. Am. Chem. Soc. 95:1328 and Lu et al. (1981) J. Org. Chem. 46:3433. Wang resin can be prepared from Merrifield resin as described hereinbelow.

Isolation and purification of the compounds and intermediates described herein can be effected, if desired, by any suitable separation or purification procedure such as, for example, filtration, extraction, crystallization, column chromatography, thin-layer chromatography, thick-layer (preparative) chromatography, distillation, or a combination of these procedures. Specific illustrations of suitable separation and isolation procedures can be had by references to the examples hereinbelow. However, other equivalent separation or isolation procedures can, of course, also be used.

Abbreviations: The following abbreviations are intended to have the following meanings:

Ac₂O acetic anhydride

DIC diisopropylcarbodiimide

DMF dimethylformamide

Fmoc fluorenylmethyloxycarbonyl

HBTU 2-(1H-benzotriazol-1-yl)-1,1,3,3-tetramethyluronium

hexafluorophosphate

HOBt 1-hydroxybenzotriazole

KOH potassium hydroxide

NEt₃ triethylamine

NMP N-methylpyrrolidone

Nv nitroveratryl

Nvoc 6-nitroveratryloxycarbonyl and other photoremovable groups

OPfp pentafluorophenyloxy

OSu N-succinimidyloxy (also known as NHS)

Py pyridine

TFA trifluoroacetic acid

THF tetrahydrofuran

TMOF trimethylorthoformate

II. General

The present invention provides methods for the large scale preparation of chemical compounds using solid phase chemistry. Three representative solid phase chemistries leading to a diketopiperazine, a tetramic acid and a β-lactam were scaled-up effectively and resulted in products with good yields and high crude purities. Affording close to 1 g of product per 1 g of Merrifield resin, the high-load resins showed high volume productivity. The advantages of the work-ups, consisting simply of resin washes, are discussed and the possibility for automation is observed. The need for low reactant and reagent equivalents results from higher reactant concentration, and it is shown that these stoichiometries can be reduced to affordable amounts.

For the scale-up of a solid phase reaction it is generally practical to start out by adapting the conditions optimized on the research scale; only occasionally was it necessary to deviate from published protocols for low-load resins. Examples are include the use of 2,6-dichlorobenzoylchloride as a coupling agent to generate high yields in esterification reactions using high-load resins and the use of dioxane as a cosolvent in the preparation of the hydroxymethylresin. But to keep costs of added reagents and reactants reasonable and to make the approach attractive for scale-up, it was imperative to reduce the large amount of reagent equivalents typically employed in solid phase synthesis. The lowest amounts of reagents necessary to reach a particular overall yield were not probed; instead, it is shown that the large excesses commonly used in solid phase chemistry are not a necessity for scale-up when using high-load resins.

When results from reactions with low-load resins were compared to the results achieved with the high-load resins, the following observations were made:

Significantly more product per unit measure of beads could be produced with high-load resins. This is a consequence of the fact that higher concentrations of reagents and building blocks can be achieved with high-load resins.

Less wash solvents were needed per unit measure of product when using high-load resin.

Smaller vessel sizes could be employed to generate a given amount of product when using high-load resins.

These findings together with the lower cost per mmol of loading indicate that the high-load Merrifield resin is a superior resin for scale-up. The results indicate that high-load Merrifield resins are attractive supports for the fast scale-up of chemistries developed for solid phase chemistry. Together with the fact that solid phase chemistry lends itself easily to automation should make this new approach a powerful new tool for the development chemist The following features make scale-up on solid phase particularly attractive:

The possibility to use established literature or research procedures developed for different supports with only minor modifications can be expected to result in a fast scale-up protocol in chemical development.

Greatly simplified work-up procedures (washes) allow reducing synthesis time dramatically. The wash-steps have furthermore the potential for automation.

Multistep-reactions can be expected to be completed in only a few days and have been shown to result generally in crude products with purities exceeding 90%.

The cost for high-load Merrifield resin to produce a product on a 100 g scale via solid phase reactions can be expected to be on the order of a few hundred dollars.

High concentrations of reactants and reagents can be employed. This allows lowering the necessary stoichiometries without significantly compromising the final product yield and purity.

The following experiments with three different solid-phase chemistries investigate the applicability of truly high-load Merrifield resins for the expedient scale-up to 100 g or more of final product.

DKP synthesis

An elegant entry into DKP's via solid phase chemistry has recently been published. See Szardenings et al. (1997) Tetrahedron 6573. Key features of this reaction sequence are the use of the Ugi multicomponent reaction and a cyclative cleavage step to liberate the product with high purity. In their studies the authors had employed PAM and Tentagel resins.

The synthesis of a DKP via Ugi-reaction using high-load hydroxymethyl resin is shown below:

[chemical structure]

The conversion of Merrifield resin 1 with a loading of 3.9 mmol/g to hydroxymethylresin 3 was carried out in two steps in a modification of a literature protocol for low-load resins. See Bodanszky et al. (1966) Chem. & Ind. 1597. In the first step the chloride was displaced with acetate in refluxing 2-methoxyethanol with 1.5 equivalents of potassium acetate. This conversion was monitored by measuring the amount of liberated chloride with an ion-selective chloride electrode. The hydrolysis of 2 to the hydroxymethylresin 3 was achieved with 2 equivalents of hydroxide in water and was monitored by IR (diminishing C═O absorption) and by nano-probe ¹H-NMR (disappearance of the acetyl methyl group). See Fitch et al. (1994) J. Org. Chem. 7955. It was found that the reaction needed dioxane as a cosolvent and elevated temperatures to proceed to completion.

[chemical structure]

The coupling of Fmoc-valine with 3 was performed using a variation of a method developed by Sieber. See Sieber (1987) Tetrahedron Lett. 6147. Using 3 equivalents of Fmoc protected valine and coupling reagents at a concentration of 0.6 M in NMP, 4 was generated in a 98% yield. Removal of the Fmoc group under standard conditions resulted in resin 5 to which were then added the starting materials for the Ugi-reaction. This multicomponent reaction was performed according to the literature procedure. The notable difference was that the reagent-equivalents employed were reduced from five equivalents to three because the concentration of reagents in this reaction was 0.8M, whereas the literature procedure had employed a concentration of 0.29M. After two acidic and one basic cyclative cleavage and a short extractive work-up, the product 7 was isolated as a 4:3 mixture of diastereomers. From 25 g of hydroxymethylresin 3 was obtained 34 g of DKP 7 (79% yield) with a crude purity of 93% (HPLC).

Tetramic acid synthesis

A solid phase synthesis on low-load polystyrene resin was recently described for 3-acyl tetrainic acids. See U.S. Ser. No. 08/896,799.

[chemical structure]

The Wang resin was chosen to allow for a simplified cleavage of intermediates for reaction monitoring. Merrifield resin 1 with a loading of 4.4 mmol/g was allowed to react with 3 equivalents of 4-hydroxybenzylalcohol in DMF at 50° C. to yield the Wang resin 8. This resulted in a quantitative displacement of chloride and a weight gain corresponding to a complete reaction. Wang resin 8 was then mixed with 3 equivalents of Fmoc-phenylalanine and DIC/HOBt in NMP at a concentration of 0.6M to yield 9. This coupling step resulted in a 55% yield. It was later found that conditions employing 2,6-dichlorobenzoylchloride result in higher yields in esterification reactions with high-load resins. The Fmoc group in 9 was removed and a reductive alkylation with 5 equivalents of anisaldehyde in trimethylorthoformate/dichloromethane 1/1 was performed. This was followed by the addition of 10 equivalents of solid sodium cyanoborohydride to yield 11. (Both aldehyde and sodium borohydride equivalents were reduced by a factor of 4 and 3 respectively compared to the literature protocol.) An extensive wash sequence with HPLC-monitoring of anisaldehyde followed to eliminate anisaldehyde as a contaminant in the final product.

The resulting secondary amine 11 was treated with acyl Meldrum's acid 12 in dioxane at 65° C. Under these conditions the Meldrum's acid was fairly unstable. The best yield of final product was generated when this acylation step was performed twice with 2.5 equivalents of 12 for 2.5 hours each time. After the acylation the addition of hydroxide in a mixture of dioxane and dichloromethane was used to induce a cyclative cleavage of the product 14 into solution. Starting with 84.7 g of Wang resin 8 (which corresponds to 61 g of 4.3 mmol/g Merrifield resin 1) the synthesis resulted in 52.1 g of the desired tetramic acid 14 with a crude purity of 95%. This corresponds to an overall yield of 40%. This yield reflects mostly the modest coupling efficiency when using HOBT/DIC to generate 9.

β-lactam synthesis

In this synthesis, published in 1996, Sasrin resin was used to allow for a mild cleavage of the product from the solid support. See Ruhland et al. (1996) J. Am. Chem. Soc. 253. The preparation of high-load Sasrin resin employed a procedure slightly modified from a recent literature protocol for low-load resin (scheme 4). See Katritzky et al. (1997) Tetrahedron Lett. 7849.

[chemical structure]

[chemical structure]

High-load 4.4 mmol/g Merrifield resin 1 was treated with 3 equivalents of 2-methoxy-4-hydroxy benzaldehyde, 3 equivalents of potassium carbonate and 0.01 equivalents of potassium iodide at 55° C. in DMF for 16 hours to yield the resin-bound aldehyde 15. Analysis of the liberated chloride with a chloride selective electrode together with the weight-gain of the resin indicated a complete reaction. The reduction of the aldehyde resin 15 was performed with 4 equivalents of sodium borohydride in a mixture of THF, N-methyl morpholine and ethanol for 16 hours to yield the high-load Sasrin resin 16. The extent of the reduction could be assessed using nano-probe ¹H-NMR: The aldehyde proton in 15 gave a distinct resonance at 10.3 ppm. This resonance disappeared in 16 and the two new benzylic protons in 16 could be clearly identified and assigned at 4.6 ppm.

The Sasrin-resin 16 was then used to perform a β-lactam synthesis (Scheme 5). The resin 16 was coupled with 3 equivalents of Fmoc-valine using 2,6-dichlorobenzoylchloride and reagent concentrations of 0.74M. The coupling was similarly effective when 2 equivalents of Fmoc-valinyl chloride were employed together with 2 equivalents of pyridine in DCM. This resulted in a quantitative coupling yield as determined by the weight gain of the resin whereas a Fmoc-quantitation showed a slightly lower yield of 95%. Any unreacted hydroxygroups in 17 were then capped in a reaction with acetic anhydride and pyridine. The Fmoc group was removed to yield 18 and the resin-bound amine was then condensed with 2.5 equivalents of benzaldehyde in a 1:1 mixture of dichloromethane and trimethylorthoformate for 3 hours. (The literature procedure uses 10-15 equivalents for this step.) The following cycloaddition reaction was performed by slowly adding 2.5 equivalents of phenoxyacetylchloride to a dichloromethane suspension of 19 together with 2.5 equivalents of triethylamine at 0° C. The reaction mixture was allowed to warm to room temperature overnight and the product was cleaved with dilute TFA in DCM. From 169 g of Sasrin resin (which is prepared from 119 g of 4.4 mmol/g Merrifield resin) was obtained 105 g of crude product 21 with a purity of >95%. After crystallization with toluene 100 g of 21 with a purity of >99% was obtained as a 2:1 mixture of diastereomers. This corresponds to an overall yield from the Merrifield resin of 56%.

Scale-up Process

For solid phase reactions on multigram scales, vessels that are based on the traditional fritted peptide vessel were used. These vessels are commercially available from vendors like Chemglass. To this design were added additional necks with ground glass joints as well as a jacket for heating and cooling. Custom made vessels were from Stanford Glass, Palo Alto, Calif. The necks serve as convenient inlet ports for the addition of reagents, for inert gas and for the introduction of a mechanical stirrer. Controlled mechanical stirring with a Teflon paddle on a glass rod using speeds up to 200 rpm did not generally affect the mechanical integrity of the beads. The bead-shape and size were monitored using a microscope.

For solid phase reactions employing volumes in excess of 1 liter the vessel design included a clamped top. The easy removal of the top resulted in a simplified sampling of the resin and allowed for convenient reaction control studies. This also greatly reduced the time to collect the whole resin batch for the purpose of drying.

The equivalent to a traditional work-up in solution phase chemistry is a wash sequence in solid phase chemistry. It serves the purpose of washing away excess reagents and reaction by-products from the resin. It was found to be advantageous to monitor the content of the washes (generally by HPLC) to reduce the amount of wash-solvents. An even more important aspect of a monitored wash is the prevention of contamination of the final product.

Table I summarizes the results in the scale-up of solid phase reactions using commercially available high-load Merrifield resins:

TABLE I

| | Merrifield | | | Crude | Wash |
|---|---|---|---|---|---|
| | volume | | | yield | purity |
| Chemistry | equivalent (g) | Linker | Vessel¹ | (g/%) | (%) |
| DKP | 28 | none | 1 liter | 34/93% | 28 |
| Tetramic acid | 61 | Wang | 2 liter | 52/95 | 23 |
| Beta-lactam | 115 | Sasrin | 2 liter | 105/95 | 42 |

¹The large-scale linker preparations were done in round-bottom flasks. For all other steps the indicated size of a modified peptide was the vessel that was used.

All reactions that were investigated were amenable to fast scale-up and each of the three syntheses was performed in 2 days or less. (This assumes that the linker has been coupled to the resin. Otherwise this step adds an additional day for Wang resin and 2 days for the Sasrin and Hydroxymethylresin.)

### EXAMPLES

The following examples are included for the purpose of illustrating the invention and are not intended to limit the scope of the invention in any manner

General. All reagents and solvents were obtained from commercial suppliers and used without further purification. The following Merrifield resins were used for the scale-up synthesis: Advanced Chemtech, 4.4 mmol/g, 100-200 mesh and Polymer Laboratories, 3.9 mmol/g, 50-100 mesh. Both suppliers produce their resins by copolymerisation of styrene with chloromethylstyrene that includes 1% divinylbenzene as crosslinker. ¹H-NMR spectra were recorded on a Varian Gemini 400 MHz instrument. Elemental analyses were carried out at UC Berkeley Chemistry Laboratories, Berkeley, Calif. Synthesis sequences were performed in modified peptide vessels except otherwise noted and contained a jacket for heating/cooling, a coarse frit and multiple necks; stirring occurred via a mechanical stirrer with a Teflon paddle on a glass rod. All reactions were stirred at speeds of <200 rpm. A single wash consisted of adding 10-100% excess of solvent over the minimal amount to swell the resin, stirring for 5 minutes and filtration under vacuum or with inert gas pressure. Wash volumes are not optimized.

Preparation of high-load resins

Hydroxymethylresin 3. A mixture of Merrifield resin 1 (80 g, 0.31 mol, available from Polymer Laboratories) and potassium acetate (46 g, 0.47 mol) was heated to reflux in 2-methoxyethanol (1 l) for 16 hours. The mixture was cooled to room temperature and the resin filtered and washed with water (3×500 ml), N-methylpyrrolidone (NMP) (3×500 ml), dichloromethane (3×500 ml) and dioxane (3×500 ml). Chloride analysis using a chloride sensitive electrode indicated a quantitative release of chloride. To the resin 2 was added dioxane (610 ml) followed by water (610 ml) and sodium hydroxide (24 g, 0.63 mol). The mixture was heated to reflux for 16 hours, after which the product-resin was filtered and washed with water (3×500 ml), NMP (3×500 ml), methanol (3×500 ml) and dichloromethane (3×500 ml). Drying in a vacuum oven at 50° C. and 50 mm Hg resulted in 72 g of white resin 3 (98% yield). The IR showed complete absence of the C═O from the intermediate acetylresin 2.

Wang resin 8. A mixture of Merrifield resin 1 (150 g, 0.66 mol, available from Advanced Chemtech, Louisville, Ky.) 4-hydroxybenzylalcohol (240 g, 1.9 mol), potassium carbonate (267 g, 1.9 mol) and potassium iodide (1 g, 0.06 mol) in DMF (1.78 liter) was stirred at 55° C. for 16 hours. The resin was filtered and washed with DMF (3×500 ml), water (4×1000 ml), NMP (2×500 ml), methanol (2×500 ml) and dichloromethane (2×600 ml). Drying in a vacuum oven at 50° C. and 50 mm Hg resulted in 202 g off-white dry resin 8 (90% yield).

Sasrin resin 16. Step i: A mixture of Merrifield resin 1 (125 g, 0.55 mol), 2-methoxy-4-hydroxybenzaldehyde (250 g, 1.6 mol), potassium carbonate (230 g, 1.6 mol) and potassium iodide (1 g, 0.06 mol) in DMF (1.78 liter) was stirred at 55° C. for 16 hours. The resin was filtered and washed with DMF (3×500 ml), methanol (3×500 ml) and with dichloromethane (3×500 ml). Drying in a vacuum oven at 50° C. and 50 mm Hg resulted in 183 g of an off-white resin 15 (96% yield). Chloride analysis using a chloride sensitive electrode had indicated a quantitative release of chloride.

Step ii: To a mixture of aldehyde resin 15 (150 g, 0.435 mol) in THF (1.5 l) were added sodium borohydride (69 g, 1.82 mol), N-methyl morpholine (1 l) and ethanol (1 l). The mixture was stirred at room temperature for 16 hours. Then water (500 ml) was slowly added. The resin was then filtered and washed with water (500 ml), 3 times with THF (500 ml), 3 times with N-methylpyrrolidone (500 ml), 3 times with methanol (500 ml) and 3 times with dichloromethane (500 ml). Drying in a vacuum oven at 50° C. and 50 mm Hg resulted in 145 g of an off-white resin 16 (97% yield).

DKP Synthesis: 1N-cyclohexyl-2-[3-benzyl-6-isopropyl-2,5-dioxo-(3S,6S)-perhydro-1-pyrazinyl]hexanamide 7

Step iii: Coupling of Fmoc amino acid. A mixture of hydroxymethylresin 3 (25 g, 97.5 mmol) and Fmoc-valine (100 g, 0.3 mol) in 500 ml NMP was stirred for 15 minutes. To the mixture was added pyridine (24 ml, 0.31 mol) and dichlorobenzoylchloride (45 ml, 0.31 mol) and stirring continued for 24 hours. The resin 4 was filtered, washed with 300 ml NMP (5×300 ml), methanol (3×300 ml) and dichloromethane (5×300 ml). It was then dried in a vacuum oven at 50° C. and 50 mm Hg for 1 day after which it weighed 56.5 g (100% yield).

Step iv: Deprotection of Fmoc. The Fmoc-deprotection was carried out by adding 500 ml of a 20% piperidine in NMP solution to the resin 4 (56.5 g, 97.5 mmol) and stirring the mixture for 30 minutes. The resin was then filtered and washed with NMP (5×500 ml), 3 times with 500 ml methanol (3×500 ml) and 5 times with 500 ml dichloromethane (5×500 ml).

Step v: Ugi reaction. To the washed resin 5 were added dichloromethane (170 ml) and valeraldehyde (29 ml, 0.27 mol). After 30 minutes of stirring a solution of Boc phenylalanine (70 g, 0.27 mol) in 170 ml methanol was added as well as cyclohexylisocyanide (33 ml, 0.27 mol) and stirring continued for 15 hours. The resin was then filtered and washed with methanol (3×500 ml), NMP (3×500 ml), methanol (3×500 ml) and then with dichloromethane (5×500 ml).

Step vi: Boc deprotection. To the resin 6 was added 500 ml of dichloromethane/TFA 1/1 and the mixture was stirred for 1 hour. The resin was then drained and washed quickly 10 times with 500 ml dichloromethane before a final wash with 250 ml acetonitrile.

Cleavage step: A solution of 1% acetic acid in acetonitrile (500 ml) was added to the resin and the mixture stirred for 3 hours. The resin was drained through the coarse filter of the vessel and the cleavage was repeated with an additional 500 ml 1% acetic acid in acetonitrile-solution. The resin was drained and washed 3 times with 250 ml acetonitrile. The wash solutions were combined with the filtrates from the cleavages. Meanwhile a solution of 4% triethylamine in acetonitrile (500 ml) was added to the resin and stirred for 5 hours. The resin was drained and washed 3 times with 250 ml acetonitrile. The wash solutions and the filtrates of the base-induced cleavages were combined with the ones from the acid-induced cleavage and evaporated. The resulting oil was dissolved in a mixture of ethyl acetate (250 ml) and water (100 ml). The water phase was separated and the organic phase was extracted twice with 1 N hydrochloric acid (100 ml) and then washed with brine (200 ml) and water (100 ml). Evaporation of the solvent resulted in 34 g of product 7 as colorless oil with 93% purity (79% yield from hydroxymethylresin 3). By HPLC (detection at 254 nm) the compound consisted of 2 diastereomers in a 4:3 ratio. ¹H-NMR (400 MHz, CDCl₃) 0.86-1.0 (m, 6H), 1.16-1.45 (m, 11H), 1.55-1.75 (m, 4H), 1.77-1.93 (m, 2H), 1.99-2.11 (m, 2H), 2.36-2.4 (m, 1H), 2.8-2.88 (m, 1H), 3.4-3.61 (m, 2H), 3.99 (d, 1H, J=3 Hz), 4.2-4.27 (m, 1H), 4.82 (t, 1H, J=7 Hz), 5.8-6.0 (broad, 2H), 7.2-7.4 (m, 5H).

Tetramic acid synthesis: 5(S)-5-benzyl-3-[1-hydroxy-2-(4-methoxyphenyl)ethylidene]-1-(4-methoxybenzyl)-2,4-azolane dione 14

Step i: Coupling of Fmoc amino acid. Wang resin was prepared according to the protocol above. The original Merrifield resin had a loading of 4.3 mmol/g and could be converted quantitatively (as judged by the weight gain of the resin) to the Wang resin. This weight gain leads to a calculated loading of 3.12 mmol/g for the Wang resin. To a mixture of Wang resin 8 (84.7 g, 0.26 mol) and Fmoc-phenylalanine (252 g, 0.75 mol) in NMP (1.25 liter) was added anhydrous hydroxybenzotriazole (101 g, 0.75 mol) and diisopropylcarbodiimide (118 ml, 0.75 mol). The mixture was stirred for 20 hours in a 5 liter 3 neck flask after which the resin was filtered, washed with NMP (3×500 ml), methanol (3×500 ml) and dichloromethane (3×500 ml). The resin 9 was dried in a vacuum oven for 16 hours at 50 mm Hg and 50° C. after which it weighed 137 g (55 % yield).

Step ii: Deprotection reaction. The Fmoc-deprotection was carried out by adding a 20% piperidine in DMF solution (625 ml) to the resin 9 (137 g, 155 mmol) The loading of the resin was determined to be 1.13 mmol/g by UV quantitation of the Fmoc group. This number corresponds to a 60% yield in the Fmoc coupling step (0.155/0.26) and stirring the mixture for 30 minutes. The resin was then filtered and washed with DMF (7×625 ml).

Step iii: Reductive alkylation. To the resin 10 was added anhydrous trimethylorthoformate (625 ml) and the mixture stirred for 10 minutes. The resin was then drained under vacuum and anisaldehyde (66.4 ml, 0.56 mol) was added directly onto the resin followed by trimethylorthoformate (300 ml), dichloromethane (300 ml) and acetic acid (6.2 ml). The resin was stirred and sodium cyanoborohydride (67 g, 1.08 mol) was added over a period of 30 minutes and stirring continued for 15 hours. The resin was then filtered under vacuum, washed with methanol (2×500 ml), DMF (3×500 ml) and dichloromethane (5×500 ml).

Step iv: Acylation step. A solution of the acyl Meldrum's acid 12 (113 g, 0.38 mol) in dioxane (630 ml) was added to the resin 11 and the mixture was stirred at 65° C. for 2.5 hours. The resin was then filtered and washed with dioxane. The above reaction was repeated with a fresh batch of 12 under the same conditions. Then the resin was filtered and washed with NMP (3×500 ml), dioxane (6×500 ml) and with a 1:1 mixture of methanol and dioxane (2×600 ml).

Step v: Cyclization. The resin 13 was transferred into a round bottom flask and a mixture of 0.1N KOH in methanol (1550 ml, 0.155 mol) and dioxane (1550 ml) was added. After 1 hour of stirring the resin was filtered. The resin was washed with a 1:1 mixture of methanol and dioxane (1 liter) and filtered. Both filtrates were combined and evaporated to dryness to yield 52.1 g of the product 14 (yield=65% from the Fmoc phe loaded resin) with a purity of 95.4% (HPLC). ¹H-NMR (400 MHz, DMSO) 2.95 (dd, 1H, J₁=4.9 Hz, J₂=14 Hz), 3.11 (dd, 1H, J₁=4.9 Hz, J₂=14 Hz), 3.38 (d, 1H, J=9.3 Hz), 3.46 (d, J=9.3 Hz), 3.75 (s, 6H), 3.94 (d, 1H, J=12.5 Hz), 4.13 (d, 1H, J=12.5 Hz), 5.04 (d, J=14 Hz), 6.8-7.3 (m, 15H). Anal. Calcd for C₂₈H₂₆KNO₅×H₂O: C, 65.48; H, 5.69; N, 2.72. Found: C, 65.55; H, 5.60; N, 2.51.

β-lactam synthesis: Cis-1-[(S)-2-(3-methyl)butanoic acid]-3-phenoxy-4-phenyl-azetiding-2-one 21

Step i: Coupling of Fmoc amino acid. To a mixture of Sasrin resin 16 (169 g, 0.49 mol) and Fmoc-valine (500 g, 1.48 mol) in NMP (2 1) were added 2,6-dichlorobenzoylchloride (211 ml, 1.48 mol) and pyridine (120 ml, 1.48 mol). The mixture was stirred in a round bottom flask for 18 hours after which it was filtered. The resin was washed with NMP (3×750 ml), methanol (3×600 ml), dichloromethane (3×600 ml) and dried which resulted in 326 g of white resin 17 (yield=100%).

Capping reaction. To the resin 17 were added acetic anhydride (800 ml) and pyridine (8 ml, 0.1 mol) and the mixture was stirred for 3 hours at 45° C. Then the resin was filtered, washed with NMP (3×750 ml), methanol (3×750 ml) and dichloromethane (3×600 ml).

Step ii: Deprotection reaction. The Fmoc-deprotection was carried out by adding a 20% piperidine in DMF solution (1000 ml) to the resin 17 and stirring the mixture for 60 minutes. The resin was then filtered and washed with NMP (3×750 ml), methanol (3×750 ml) and dichloromethane (3×750 ml).

Step iii: Imine formation. To the resin 18 were added dichloromethane (650 ml)), anhydrous trimethylorthoformate (650 ml) and benzaldehyde (122 ml, 1.2 mol) and the mixture was stirred at room temperature for 3 hours. It was then filtered and washed with NMP (3×750 ml), methanol (3×750 ml) and dichloromethane (3×600 ml).

Step iv: Cycloaddition. To the resin 19 was added dichloromethane (650 ml) and the mixture was cooled to 0° C. To the mixture was added triethylamine (163 ml, 1.18 mol). A solution of phenoxyacetylchloride (163 ml, 1.18 mol) in dichloromethane (200 ml) was then added over a period of 30 minutes with the temperature at 0° C. After the addition of the chloride the temperature was raised to room temperature over a period of 2 hours and the mixture stirred for an additional 12 hours. It was then filtered and washed with NMP (3×750 ml), methanol (3×750 ml) and dichloromethane (3×600 ml).

Step v: Cleavage. A 9:1 mixture of dichloromethane and trifluoroacetic acid (1000 ml) was added to the resin 20, and the mixture was stirred for 1 hour. The resin was then filtered and washed with dichloromethane (1000 ml). This wash-eluent was combined with the cleavage-filtrate and the solution evaporated to dryness. This resulted in an oil (105 g) with a purity of 95% (HPLC). The addition of toluene (1000 ml) caused the product to solidify and triturating with hexane yielded 21 as a white solid with a purity of >99% by HPLC (100 g, yield =60% from Sasrin resin). ¹H-NMR (400 MHz, CDCl₃) 2.5:1 mixture of 2 cis diastereomers; Major isomer: 0.91 (d, 3H, J=6.9 Hz), 0.95 (d, 3H, J=6.9 Hz), 2.15-2.12 (m, 1H), 4.11 (d, 1H, J=6.95 Hz), 5.22 (d, 1H, J=4.7 Hz), 5.54 (d, 1H, J=4.7 Hz), 7.13-6.72 (m, 5H), 7.42-7.25 (m, 5H). Minor isomer: 1.02 (d, 3H, J=6.6 Hz), 1.18 (d, 3H, J=6.6 Hz), 2.72-2.63 (m, 1H), 3.68 (d, 1H, J=8.15 Hz), 4.96 (d, 1H, J=4.8 Hz), 5.48 (d, 1H, J=4.7 Hz), 7.13-6.72 (m, 5H), 7.42-7.25 (m, 5H). Anal. Calcd for C₂₀H₂₁NO₄: C, 70.78; H, 6.24; N, 4.13. Found: C, 70.68; H, 6.30; N, 3.75.

The above description is illustrative and not restrictive. Many variations of the invention will become apparent to those of skill in the art upon review of this disclosure. Merely by way of example a wide variety of process times, reaction temperatures, and other reaction conditions may be utilized, as well as a different ordering of certain processing steps. The scope of the invention should, therefore, be determined not with reference to the above description, but instead should be determined with reference to the appended claims along with their full scope of equivalents. Although certain embodiments and examples have been used to describe the invention, changes may be made to those embodiments and examples without departing from the scope of the following claims or spirit of the invention.

The disclosures in this application of all articles and references, including patent documents, are incorporated herein by reference.

What is claimed is:

1. In a method of synthesizing an organic compound in multigram scale, the improvement comprising synthesizing such compound on Merrifield resin.

2. The method of claim 1, wherein the Merrifield resin has a loading of about 3-6 millimoles per gram.

3. In a method of synthesizing esters in the solid phase on high-load resins, the improvement of using 2,6-dichlorobenzoylchloride as a coupling agent.

4. In a method of preparing hydroxymethylresin, the improvement of using aqueous dioxane as cosolvents.

5. The method of claim 3, wherein the ratio of water to dioxane is about 1:1.
